(19) United States
(12) Patent Application Publication
(10) Pub. No.: US 2025/0134542 A1
(43) Pub. Date: May 1, 2025

(54) SURGICAL APPARATUS WITH DISTAL JAW AND IMAGING FEATURES

(71) Applicant: Arthrex, Inc., Naples, FL (US)

(72) Inventors: Loren Crook, Fort Myers, FL (US); John Sodeika, Naples, FL (US); Ryan Kellar, Naples, FL (US); Thomas Dooney, Naples, FL (US); Jason Valentin, Fort Myers, FL (US); Joshua Dines, Southampton, NY (US)

(73) Assignee: Arthrex, Inc, Naples, FL (US)

(21) Appl. No.: 18/924,145

(22) Filed: Oct. 23, 2024

Related U.S. Application Data
(60) Provisional application No. 63/545,856, filed on Oct. 26, 2023.
(60) Provisional application No. 63/687,008, filed on Aug. 26, 2024.

(51) Int. Cl. A61B 17/29; A61B 1/00; A61B 17/04; A61B 17/06

(52) U.S. Cl. CPC A61B 17/29; A61B 1/00087; A61B 17/0483; A61B 17/06066

(57) ABSTRACT

A surgical apparatus includes an elongated probe extending from a proximal handle portion to a distal jaw. In operation, the distal jaw may be actuated via an actuator assembly in connection with the handle portion. The distal jaw may be utilized to manipulate or grasp patient tissue, sutures, or various objects associated with a procedure that may be encountered in a patient cavity. An imaging device is in connection with the probe proximal to the distal jaw. In operation, the imaging device may capture image data in a field of view distally directed from the elongated probe. The image data captured in the field of view may assist an operator in targeting objects to be grasped or clamped within the distal jaw while avoiding unintentional contact with neighboring tissue within the patient cavity.

### CROSS-REFERENCE TO RELATED APPLICATIONS

This application claims priority under 35 U.S.C. § 119 (e) and the benefit of U.S. Provisional Application Nos. 63/545,856 entitled SURGICAL APPARATUS WITH DISTAL JAW AND IMAGING FEATURES, filed on Oct. 26, 2023, by Crook et al. and 63/687,008 entitled SURGICAL APPARATUS WITH DISTAL JAW AND IMAGING FEATURES, filed on Aug. 26, 2024, by Crook et al., the entire disclosures of which are incorporated herein by reference.

### BACKGROUND

Minimally invasive procedures generally provide for improved patient outcomes by limiting tissue damage necessary to access a surgical site. To practice such procedures, various devices may be implemented with elongated probes to prepare and treat tissue as well as implant surgical constructs. The disclosure provides for improvements for practicing minimally invasive procedures as provided in the following detailed description.

### SUMMARY

In various implementations, the disclosure provides for a surgical apparatus that may include an elongated probe extending from a proximal handle portion to a distal jaw. In operation, the distal jaw may be actuated via an actuator assembly in connection with the handle portion. In this configuration, the distal jaw may be utilized to manipulate or grasp patient tissue, sutures, or various objects associated with a procedure that may be encountered in a patient cavity. In various implementations, an imaging device may be in connection with a portion of the distal jaw. In operation, the imaging device may capture image data in a field of view distally directed from the elongated probe. The image data captured in the field of view may assist an operator in targeting objects to be grasped or clamped within the distal jaw while avoiding unintentional contact with neighboring tissue within the patient cavity.

In various implementations, the surgical apparatus may correspond to a suture passer including a retractable needle that is selectively extended between inner grasping surfaces of the distal jaw. The distal jaw may include a fixed jaw extending from the elongated probe and a mandible jaw that may open and close at a jaw angle relative to a fixed jaw via a linkage or hinge assembly. In such implementations, the imaging device may be in connection with an outer jaw surface of the elongated probe or the fixed jaw opposite the inner grasping surface. In operation, the image data captured in the field of view may demonstrate regions that may be hidden behind patient tissue and otherwise occluded from views provided by traditional surgical scopes. In this way, the disclosure may provide for improved operation of the surgical apparatus by presenting image data demonstrating otherwise occluded regions of patient cavities or tissue associated with various procedures.

In some implementations, the disclosure may provide for a method of passing a suture through tissue relative to a feature or landmark in a patient cavity. The method may begin by inserting a distal jaw of an elongated probe into a patient cavity. Once deployed in the patient cavity, a feature in connection with or associated with a patient anatomy may be viewed from an outer jaw surface of the distal jaw in a field of view captured by an imaging device. Based on image data captured in the field of view, the elongated probe may be positioned in the patient cavity aligning a marker on the distal jaw with the feature. Once aligned, a suture passing needle may be deployed through patient tissue that is captured between inner jaw surfaces of the distal jaw. The suture passing needle extends between the inner jaw surfaces and is aligned with the marker opposite the outer jaw surface. In this way, the image data captured by the imaging device may be utilized to align the suture passing needle with the landmark in the patient cavity.

These and other features, objects and advantages of the present disclosure will become apparent upon reading the following description thereof together with reference to the accompanying drawings.

### BRIEF DESCRIPTION OF THE DRAWINGS

FIG. 1 is a projected view of a patient anatomy demonstrating a surgical apparatus;

FIG. 2A is a detailed, partial sectional view of a patient cavity demonstrating a surgical apparatus utilized for a tendon repair;

FIG. 2B is a projected view of a surgical construct demonstrating a result of a tendon repair from the procedure of FIG. 2A;

FIG. 3 is a simplified detailed view demonstrating the operation of the surgical apparatus relative to a surgical scope;

FIG. 4A is a side profile view demonstrating the surgical apparatus in the form of a suture passer;

FIG. 4B is a detailed side profile view demonstrating a distal jaw of the surgical apparatus;

FIG. 4C is a detailed front projected profile view demonstrating a distal jaw of the surgical apparatus;

FIG. 5A demonstrates an example of image data captured in a field of view of an imaging device in connection with a surgical apparatus;

FIG. 5B demonstrates an example of image data captured in a field of view of an imaging device in connection with a surgical apparatus;

FIG. 6 is a partial schematic view demonstrating an exemplary implementation of the imaging head of the surgical apparatus;

FIG. 7A is a side view of an imaging device for a suture passer;

FIG. 7B is a projected view of an imaging head for a suture passer;

FIG. 8A is a projected view of an elongated surgical device comprising a camera apparatus flexibly positioned relative to a distal jaw;

FIG. 8B is a projected view of the elongated surgical device of FIG. 8A with the rotation angle of the camera apparatus adjusted relative to the distal jaw;

FIG. 8C is a projected view of the elongated surgical device of FIG. 8A with the camera apparatus translated and rotated relative to the distal jaw; and

FIG. 9 is a schematic block diagram of a surgical imaging system in accordance with the disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

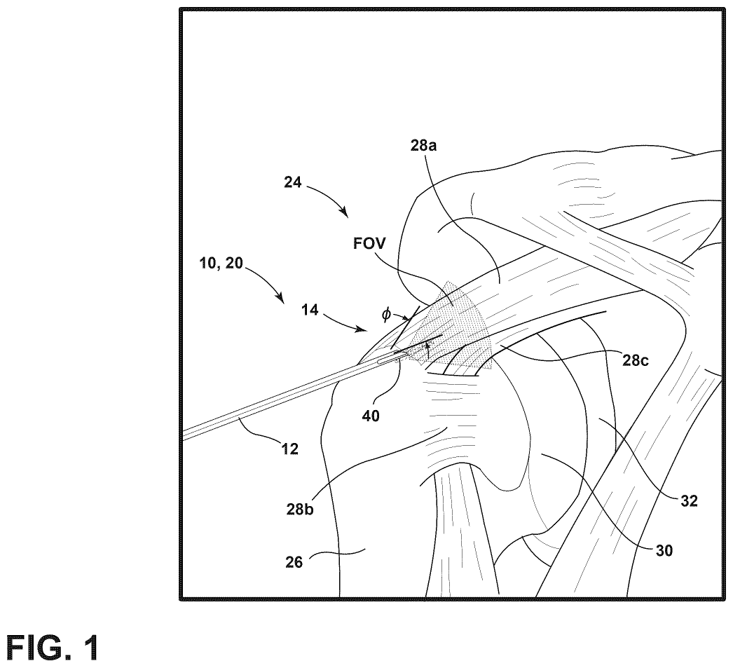

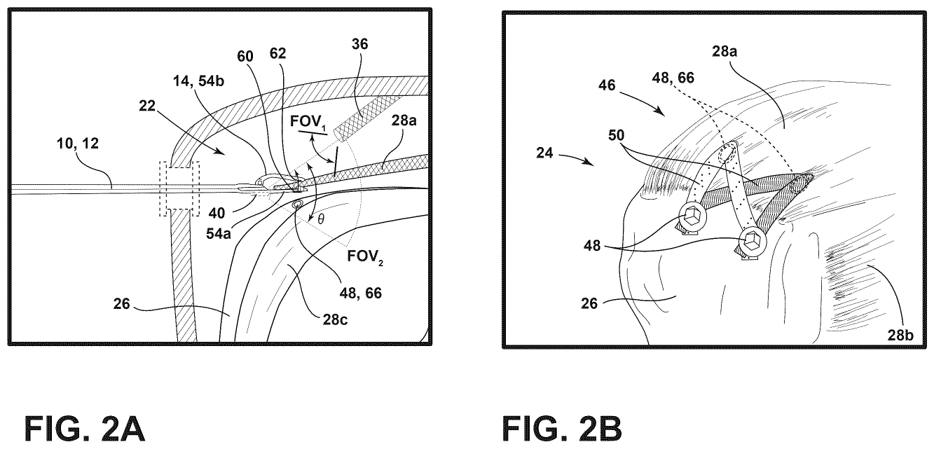

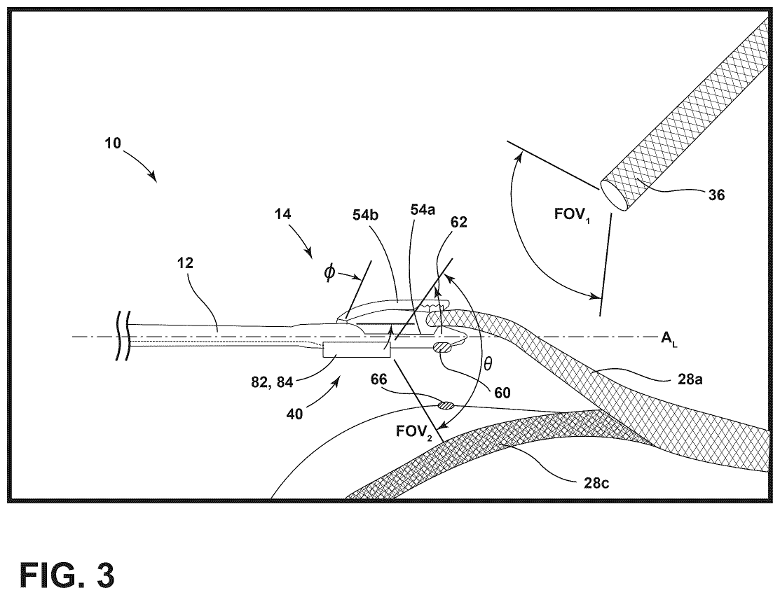

Referring generally to FIGS. 1-4, a surgical apparatus 10 is shown including an elongated probe 12 forming a distal jaw 14 extending from a proximal handle portion 16. In the example shown, the surgical apparatus 10 may correspond to a suture passer 20 deployed in a patient cavity 22. The elongated probe 12 and distal jaw 14 are shown deployed to perform a rotatory cup repair of a glenohumeral joint 24. More specifically, the surgical apparatus 10 is shown deployed between a connection of the humerus 26 to a first tendon 28a (supraspinatus tendon). As further demonstrated for reference, the glenohumeral joint 24 is demonstrated in relation to the humeral head 30, the glenoid 32, a second tendon 28b (e.g., the subscapularis tendon), and a third tendon 28c (e.g., the bicep tendon). As shown in FIGS. 2 and 3, the patient cavity 22 and anatomical features may be exaggerated in proportion and adjusted in perspective, such that the enclosed features of the surgical apparatus 10 and the patient anatomy may be clearly represented.

As exemplified in FIGS. 1, 2A and 3, in various procedures, a portion of the distal jaw 14 may be occluded behind patient tissue. In the example shown, a portion of the distal jaw 14 is obscured or occluded behind the first tendon 28a relative to a first field of view FOV₁ of a surgical scope 36 (e.g., arthroscope, endoscope, etc.). In such cases, the relative proportions and position (e.g., distal extent, depth, etc.) of the distal jaw 14 may be occluded from the first field of view FOV₁, such that the operation of the surgical apparatus 10 is partially or completely blind. In various implementations, the disclosure may provide for an imaging device 40 in connection with a portion of the distal jaw 14 that captures image data in a second field of view FOV₂ that may be distally directed from the elongated probe 12. In operation, the image data captured in the second field of view FOV₂ by the imaging device 40 may improve visibility of occluded features and landmarks corresponding to surgical constructs and/or anatomical features to guide the positioning of the elongated probe 12 and improve the operation of the apparatus 10.

Referring now to FIGS. 2A, 2B, and 3, the operation of the surgical apparatus 10 is described in reference to an exemplary surgical procedure repairing a tear to the first tendon 28a (e.g., the supraspinatus tendon). As demonstrated in FIG. 2B, the repair of the first tendon 28a may be effectuated by installing a surgical construct 46 comprising a plurality of anchors 48 or fixation devices that may interconnect the bony tissue of the humerus 26 to a separated or damaged portion of the first tendon 28a. As shown, the first tendon 28a is connected to the anchors 48 via a plurality of sutures 50 (e.g., suture tape, FiberTape® or TigerTape™) that position the separated portion of the first tendon 28a in connection with the humerus 26 to effectuate the rotator cup repair. The exemplary surgical construct demonstrated in FIG. 2B may correspond to a SpeedBridge® double-dash row technique for rotator cup repair that implements the anchors 48 as SWIVELOCK® anchors as provided by Arthrex, Inc. As shown, one or more of the anchors 48 may be hidden or occluded behind the patient tissue, in this case the first tendon 28a, such that the alignment with the corresponding sutures 50 may be occluded from the first field of view FOV₁ of the scope 36. As demonstrated in FIGS. 2A and 3, the second field of view FOV₂ may capture image data for presentation to a surgeon or operator of the surgical apparatus 10 to align a position of each of the sutures 50 with the corresponding anchors 48 even in cases where the anchor 48, feature, or landmark is occluded from the first field of view FOV₁.

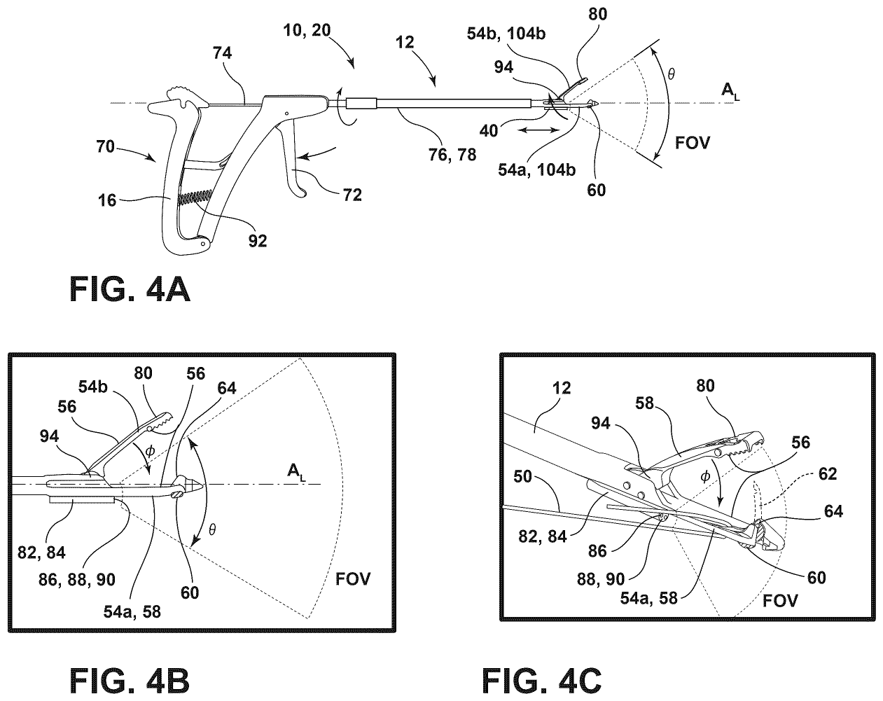

As best demonstrated in FIGS. 3 and 4, the second field of view FOV₂ may be aligned with a longitudinal axis A_{L} of the elongated probe 12. In various implementations, the distal jaw 14 may comprise a fixed jaw 54a and a mandible jaw 54b. In operation, an actuator assembly 70 in connection with the handle portion 16 of the surgical apparatus 10 may be configured to selectively open and close the mandible jaw 54b relative to the fixed jaw 54a. In response to the actuation, opposing inner grasping surfaces 56 of the jaws 54 may enclose about and engage objects, such as the first tendon 28a. The imaging device 40 may be connected to a portion of the distal jaw 14 aligned with or positioned on the fixed jaw 54a, such that the second field of view FOV₂ may remain stationary relative to the longitudinal axis A_{L} of the elongated probe 12 throughout the engagement of the mandible jaw 54b over a jaw angle ϕ. In this way, the image data captured in the second field of view FOV₂ may be representative of an alignment of the distal jaw 14 within the patient cavity 22 regardless of the jaw angle ϕ and corresponding position of the mandible jaw 54b. Though described as being aligned with the longitudinal axis A_{L} of the elongated probe 12, the second field of view FOV₂ may similarly be angled at various angles ranging from 0° to 180° (or proximally from distal jaw 14) depending on the desired orientation of the second field of view FOV₂. For example, the second field of view FOV₂ may be directed at 0°, 10°, 15°, 30°, 45°, 60°, 90°, 120°, 180°, or any angular orientation therebetween relative to the longitudinal axis A_{L}.

Still referring to FIG. 3, in the example shown, the mandible jaw 54b is closed grasping a cross-section of the first tendon 28a in the inner grasping surface 56 in coordination with the fixed jaw 54a. The imaging device 40 may be in connection with an outer jaw surface 58 of the fixed jaw 54a opposite the inner grasping surface 56. In various implementations, a marker 60 may be positioned along the outer jaw surface 58 in alignment with an operating feature of the surgical apparatus 10, in this case a retractable needle 62 of the suture passer 20. The marker 60 may correspond to a protrusion, undulation, surface feature, and/or colored, etched, or illuminated indicator positioned on the outer jaw surface 58. In the example of the retractable needle 62, the marker 60 may be positioned on the outer jaw surface 58 in alignment with a needle aperture 64. As shown in FIG. 4C, the needle aperture 64 may be formed on the inner grasping surface 56 through which the retractable needle 62 extends to pass the suture 50 from the fixed jaw 54a to the mandible jaw 54b. In this configuration, the marker 60 may be aligned with a feature 66 or landmark and may be indicative of a position of the distal jaw 14 relative to the anatomy of the patient in the patient cavity 22. In the example shown, the marker 60 is aligned with one of the anchors 48 hidden behind the first tendon 28a relative to the first field of view FOV₁. Accordingly, the image data captured in the second field of view FOV₂ may be reviewed by a user of the surgical apparatus 10 to align the retractable needle 62 and the resulting position of the suture 50 in the first tendon 28a in alignment with the feature 66 or anchor 48 to ensure accurate placement of the suture 50.

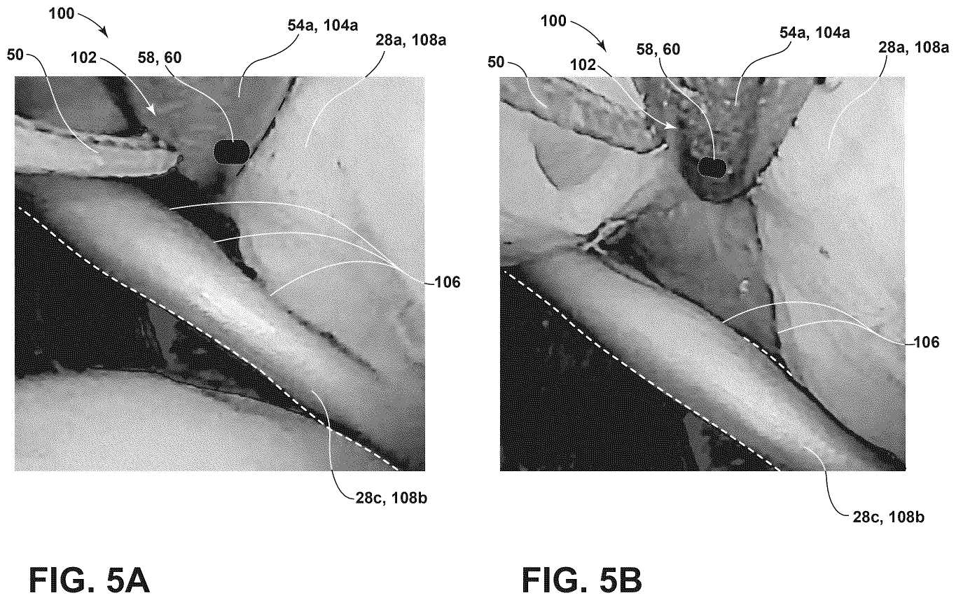

In addition to accurately positioning the suture 50, the image data presented in the second field of view FOV₂ and captured by the imaging device 40 may be monitored by a user to ensure that neighboring tissue is not captured by the distal jaw 14 and/or penetrated by the retractable needle 62. As best demonstrated in FIGS. 1 and 2A, the first tendon 28a is demonstrated passing adjacent to the third tendon 28c (e.g., the bicep tendon). Though demonstrated as having a significant clearance between the first tendon 28a and the third tendon 28c, in some cases, the adjacent tendons (e.g., tendons 28a, 28c) may pass closely to each other within the patient cavity 22. In such cases, the conventional blind operation of the distal jaw 14 may create uncertainty as to the extent of the patient tissue (e.g., the first tendon 28a, the third tendon 28c, etc.) captured within the inner grasping surfaces 56 of the distal jaw 14. By reviewing the image data captured in the second field of view FOV₂, a user may ensure that an extent of the tissue grasped within the grasping surfaces 56 does not extend to neighboring tissue or other unexpected objects that may be present in the patient cavity 22. In this way, the surgical apparatus 10 may provide for improved accuracy in the placement of the sutures 50 as well as the engagement of the target tissue within the distal jaw 14. Annotated representations of actual image data 100 are shown in FIGS. 5A and 5B and clearly demonstrate the importance of distinguishing the target tissue from the neighboring tissue as discussed herein.

Referring now to FIGS. 4A, 4B, and 4C, the surgical apparatus 10 is shown demonstrating several features related to the operation of the suture passer 20 and the imaging device 40. As demonstrated in FIG. 4A, the actuator assembly 70 is shown in connection with the handle portion 16. In operation, the actuator assembly 70 may control the closure of the mandible jaw 54b adjusting the jaw angle ϕ in response to the depression of a trigger 72. The compression of the trigger 72 may engage the actuator assembly 70 causing an actuation linkage 74 to translate in a conduit 76 formed within a probe body 78 of the elongated probe 12. In operation, the translation of the actuator linkage 74 may result in the closure of the mandible jaw 54b causing the jaw angle ϕ to vary from an open position (e.g., FIG. 4B) to a closed or clamped position (e.g., FIG. 2A). Following the closure of the distal jaw 14, the retractable needle 62 is forced outward through the needle aperture 64 by the actuator linkage 74 and into a suture clamp 80 of the mandible jaw 54b. In this way, the suture 50 may be passed from the fixed jaw 54a to the mandible jaw 54b and through any intervening tissue captured within the inner grasping surface 56 of the distal jaw 14 in the clamped configuration.

As previously discussed, the imaging device 40 may be positioned along a portion of the elongated probe 12 proximate to the distal jaw 14. In the example shown, the imaging device 40 is implemented utilizing control circuitry 82 incorporated in an imaging head 84 disposed in a distal end portion of the elongated probe 12. In this configuration, light may be output from one or more emitters 86 (e.g., light emitting diodes) disposed at a distal end portion 88 of the imaging device 40. The light emitted from the emitters 86 may be reflected within the patient cavity 22 and may be captured by image sensors 90 (e.g., complementary metal oxide semiconductor [CMOS] sensors) positioned on the distal end portion 88. In this configuration, the image sensors 90 may detect and process the light impinging on the distal end portion 88 of the imaging head 84 and communicate the resulting image data to a display controller via a communication interface. In various implementations, the communication interface may correspond to a wired or wireless communication interface that may extend through the elongated probe 12 to the handle portion 16.

As previously discussed, the image data captured via the image sensor 90 of the imaging device 40 may be observed to determine an alignment between the retractable needle 62 and the feature 66 or landmark in the patient cavity 22 based on the position of the marker 60. The marker 60 may be positioned along the outer jaw surface 58 in alignment with a deployment path of the retractable needle 62 or various actuating features. The marker 60 may correspond to a protrusion, undulation, surface feature, and/or colored or illuminated indicator positioned on the outer jaw surface 58 in alignment with the needle aperture 64 formed on the inner grasping surface 56. In operation, the engagement of the actuator assembly 70 may cause the retractable needle 62 to pass the suture 50 from the fixed jaw 54a to the suture clamp 80 formed in the mandible jaw 54b. By viewing the position of the marker 60 relative to the features 66 or landmark in the patient cavity, the position of the retractable needle 62 may be accurately inferred, thereby ensuring accurate positioning of the suture 50.

Once the suture 50 is passed from the fixed jaw 54a to the mandible jaw 54b, the suture 50 may be captured in the suture clamp 80 formed in the mandible jaw 54b. In this configuration, the suture 50 may be withdrawn from the patient cavity 22 or otherwise positioned to effectuate the surgical procedure (e.g., tied, secured, passed, etc.). The opening of the mandible jaw 54b and the retraction of the retractable needle 62 may be driven by the extension of a biasing spring 92 that may be in connection with the actuator assembly 70 and the handle portion. In this configuration, the distal jaw 14 may be closed, decreasing the jaw angle ϕ in response to the force applied to the trigger 72, and may be opened, increasing the jaw angle ϕ in response to the force applied by the biasing spring 92.

As best illustrated in FIG. 4B, a camera axis of the imaging device 40 may be aligned with the longitudinal axis A_{L}, and the distal end portion 88 may be position proximate to a hinge assembly 94 of the distal jaw 14. In various implementations, the distal end portion 88 of the imaging device 40 may be positioned such that the second field of view FOV₂ expands relative to the longitudinal axis A_{L} at the viewing angle θ, such that the marker 60 and the feature 66 or landmark indicating the desired position of the suture 50 are visible in the second field of view FOV₂. Accordingly, the position of the imaging device 40 in connection with the elongated probe 12 may be adjusted to ensure that the viewing angle θ captures the relevant features 66 or landmarks (e.g., anatomy, anchors, constructs, surgical implements, etc.) necessary to align the surgical apparatus 10 to effectuate a variety of procedures. Accordingly, depending on the viewing angle θ and the distance commonly separating the marker 60 from the feature or landmark in a given procedure, the longitudinal position of the distal end portion 88 of the imaging device 40 may be repositioned proximally or distally to capture the relevant information (e.g., features 66 or landmarks) in the image data.

In various implementations, the imaging head 84, including the image sensor(s) 90 and the one or more emitters 86, may be deployed in a cylindrical package forming the imaging head 84 with a longitudinal profile shape having approximate dimensions of less than 2 mm in diameter and less than 6 mm in length with a corresponding resolution of 400×400 pixels and a viewing angle θ of 120° or more (e.g., 130°, 160°, etc.). In general, the resolution of the image sensor 90 may be 200×200, 400×400, 800×800 or more. In various cases, the image sensor 90 may form a package that may have a width less than 2 mm, less than 1 mm, or approximately from 0.5-0.7 mm. Additionally, the image sensor 90 may be arranged having a height less than 2 mm, less than 1 mm, or approximately from 0.5-0.7 mm. Though shown and described in reference to the imaging head 84 being cylindrical in shape, the imaging head 84 including the image sensor(s) 90 and the one or more emitters 86 may similarly be implemented in an elliptical, rectangular, or square longitudinal profile shape as well as various complex geometries that may conform to a shape of a portion of the surgical apparatus 10. For example, the longitudinal profile shape of the package forming the imaging head, including the image sensor(s) 90 and the one or more emitters 86, may be deployed in a package having an approximate width of less than 2 mm, 1.5 mm, or 1 mm and an approximate height of less than 2 mm, 1.5 mm, or 1 mm. Accordingly, the imaging device 40 may be incorporated in various arrangements to suit the operating requirements of the surgical instrument 10.

Referring now to FIGS. 5A and 5B, sample image data 100 is shown demonstrating a distal end portion 102 of the distal jaw 14 as captured in the second field of view FOV₂ of the imaging device 40. A ventral surface 104a of the outer jaw surface 58 extends opposite a dorsal surface 104a (see FIG. 4) and is shown in FIGS. 5A and 5B extending centrally within the second field of view FOV₂. In this configuration, a plurality of tissue boundaries 106 between a target tissue 108a (e.g., the first tendon 28a) and an adjacent or neighboring tissue 108b (e.g., the third tendon 28c) may clearly be visible within the second field of view FOV₂ in relation to a grasping extent of the distal jaw 14. Based on the image data 100, the tissue boundaries 106 that were previously discussed as being occluded in the first field of view FOV₁ of the surgical scope 36 may be readily identified in the image data 100. Accordingly, the surgeon or operator of the surgical apparatus 10 may selectively actuate the distal jaw 14 with a clear visual indication in the image data 100 that the neighboring tissue 108b is outside an engagement or grasping extent of the distal jaw 14 and the target tissue 108a is within the inner grasping surfaces 56 of the distal jaw 14. Without the image data 100, the determination of the clearance of the neighboring tissue 108b or untargeted tissue may have to be blindly determined leading to uncertainty in clearance and positioning of the surgical device 10 and the suture(s) 50.

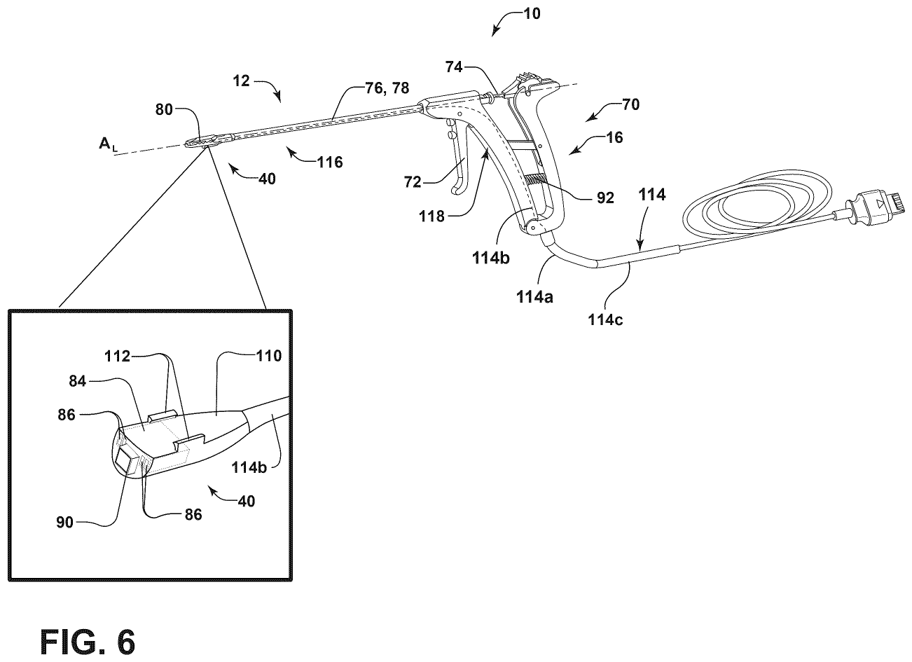

FIG. 6 is a partial schematic view demonstrating an exemplary implementation of the imaging head 84 of the surgical apparatus 10. In various implementations, the imaging head 84 may be enclosed within a detachable, elongated low-profile housing 110. In this configuration, the imaging device 40 may be implemented as an optional assembly associated with the surgical apparatus 10. In some implementations, the housing 110 may include one or more connecting features 112 that may be configured to interconnect the housing 110 to a distal end portion of the elongated probe 12. In the example shown, the connecting features 112 correspond to mating tabs or protrusions that may engage corresponding openings formed in the distal end of the elongated probe 12. Though a specific example of the connecting features is shown, it shall be understood that various features and connecting interfaces may be implemented to interconnect the housing 110 with the elongated probe 12.

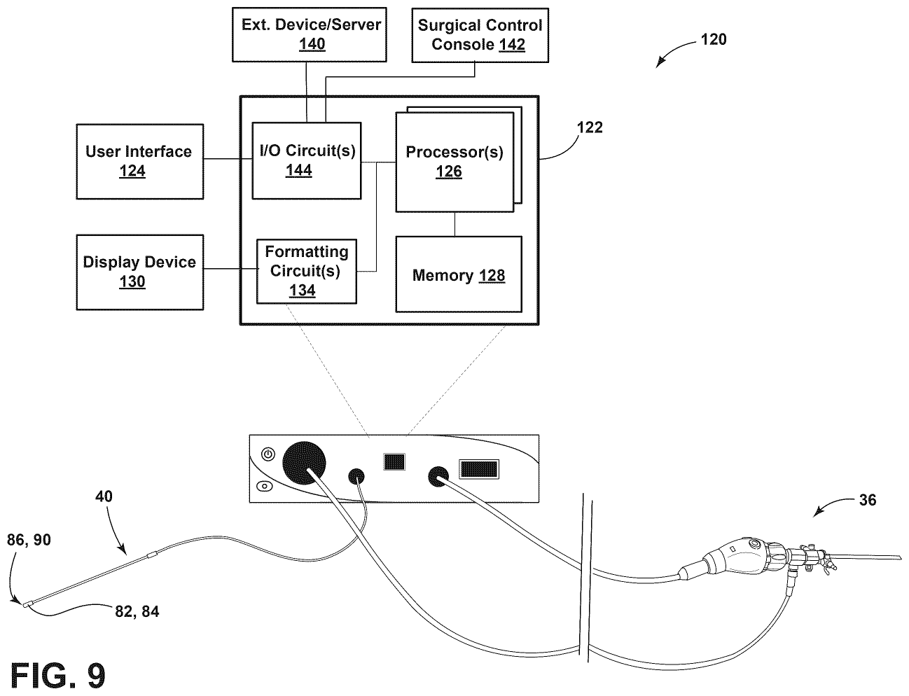

In various implementations, the imaging device 40 may be in communication with a controller or control console via a communication cable 114. As shown in FIG. 6, the elongated probe 12 may form an elongated trough 116 or cannula extending from a proximal end portion 12a to a distal end portion 12b of the elongated probe 12. Further, the elongated trough 116 or cannula of the probe 12 may interconnect with an interior passage 118 that may extend through a body of the handle portion 16 to a cable interface interconnecting the communication cable 114 to a proximal end portion of the handle 16. In this configuration, the communication cable 114 may form an extension cable interconnecting the imaging device 40 to a corresponding controller. As shown, opposite the connection to the surgical instrument 10, the communication cable 114 includes a connection interface configured to communicatively couple the imaging device 40 to a corresponding controller. A detailed description of the controller and control system associated with the imaging device 40 is further described in reference to FIG. 9.

In some implementations, the communication cable 114 may include a cable management feature that may limit strain while also improving the usability of the surgical instrument 10. As shown in FIG. 6, the communication cable 114 may be connected to the handle portion 16 via a strain relief boot 114a. In addition to the strain relief boot 114a, the communication cable 114 may comprise a first cable 114b portion extending through the handle portion 16 of the actuator assembly 70 as well as a second cable portion 114c extending from the surgical instrument and communicatively connecting the surgical instrument with the controller 142 of the imaging system 140 (FIG. 9). In this configuration, the strain relief boot 114a may support the communication cable 114 extending away from the handle portion 16 to avoid interference with a surgeon or operator engaging the instrument 10. In various implementations, the second cable portion 114c may correspond to a lighter, thinner, and/or smaller gauge communication cable and sheathing or jacket than the first cable portion 114b. In this way, the second cable portion 114c interconnecting the surgical instrument 10 with the controller 142 may limit a pulling force or tension along the second cable portion 114c, which may encumber maneuverability of the surgical instrument 10 or otherwise increase an effective weight of the surgical instrument 10. By incorporating the strain relief boot 114a or reinforcement feature, the first and second cable portions 114b, 114c may limit improve the maneuverability of the surgical instrument 10 while maintaining a robust communicative connection to the controller 142.

Still referring to FIG. 6, the detailed view of the housing 110 demonstrates the low profile arrangement of the image sensor 90, including a plurality of emitters 86 positioned on opposing sides of the distal end portion 88. In this arrangement, the image sensor 90 and emitters 86 may be positioned adjacent to the elongated probe 12 and interconnected thereto by the structure forming the housing 110. In this configuration, the adjacent positioning of the emitters 86 and image sensor 90 relative to the elongated probe 12 may limit a clearance dimension associated with a protrusion length of the low-profile housing 110 extending outward from the elongated probe 12 transverse to the longitudinal access of the probe 12.

In various implementations, the image sensor 90 may correspond to a micro camera module that integrates the emitters 86 in a package assembly. The proportions of the overall package assembly may be approximately 5 mm or less in width and 3 mm or less in height, where the height corresponds to the direction protruding from the ventral surface the elongated probe 12, opposite the mandible jaw 54b. In some implementations, the lateral dimensions of the imaging device 40 relative to the longitudinal axis A_{L} may be less than 3 mm in width and 2 mm in height, and in some cases may be less than 2.5 mm in width and less than 1.5 mm in height. In various implementations, the resolution of the image sensor 90 may be in excess of 200×200 pixels and may exceed 400×400 pixels. Further, the field of view of the imaging device 40 may be extend over a viewing angle θ of 60°, 90°, 120° or greater including various intermediate viewing angle θ between those specifically described. Accordingly, the imaging device 40 may be implemented in a variety of packages and positioned within the low-profile housing 110 to provide for a modular imaging accessory that may be implemented as an optional accessory for the elongated probe 12 of the surgical apparatus 10.

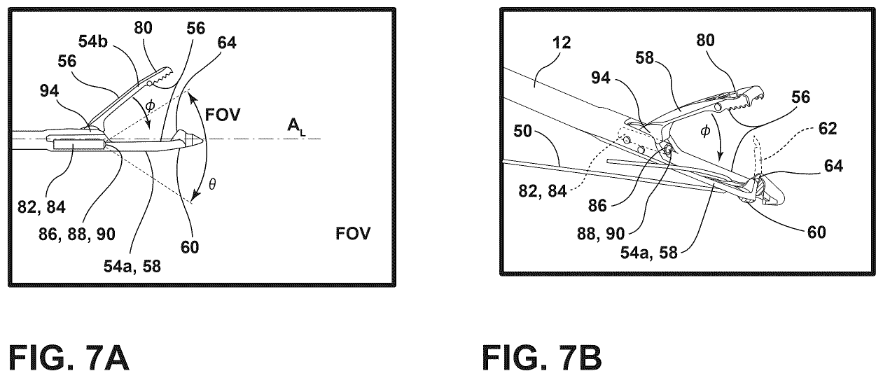

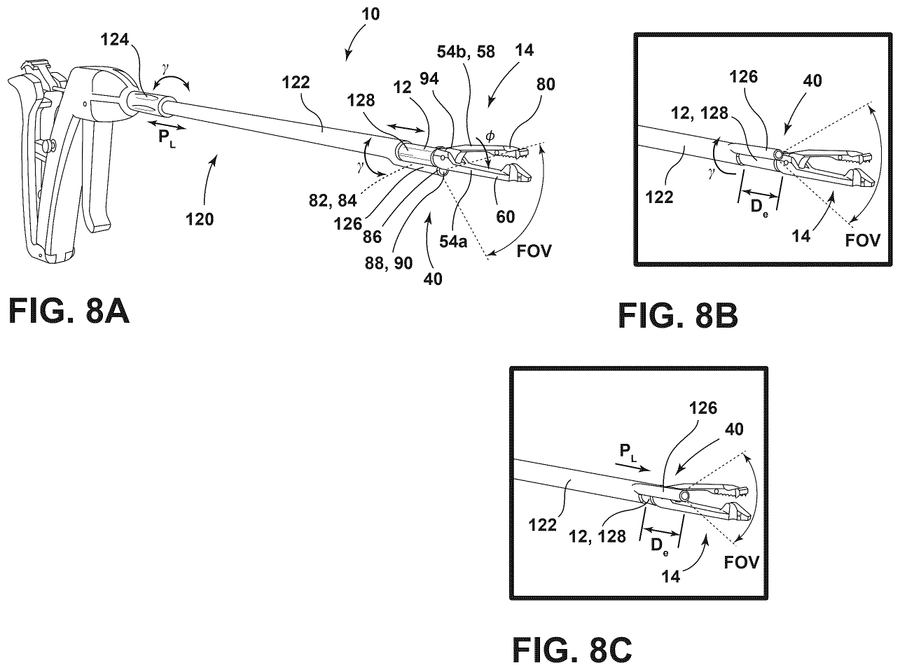

Referring now to FIGS. 7A, 7B, 8A, 8B, and 8C; the imaging device 40 and surgical apparatus 10 are described demonstrating additional features that may be implemented in various combinations depending on the associated application and user preferences. While previous examples of the surgical apparatus 10 include the imaging device 40 positioned on the ventral surface, the imaging device 40 may be implemented in various positions, orientations, and combinations in connection with the suture passer 20. As demonstrated in FIG. 7A, the imaging device 40 is positioned along a side portion with the field of view FOV generally directed between the mandible jaw 54b and the fixed jaw 54a along the longitudinal axis A_{L}. In this configuration, the field of view FOV may be focused on the path of the mandible jaw 54b adjusting the jaw angle ϕin response to the depression of a trigger 72. As shown in FIG. 7B, the imaging device may be positioned in connection with the fixed jaw 54a and adjacent to the joint or hinge assembly 94 connecting the mandible jaw 54b to the fixed jaw 54a. In this configuration, the field of view FOV may be positioned centrally between the jaws 54 without portions of the distal jaw 14 extending between the jaws 54 and the field of view FOV of the imaging device 40. Though demonstrated as individual cameras, each of the imaging devices 40 provided may be implemented in various combinations or in interchangeable positions or orientations relative to the distal jaw 14 to provide improved visualization. Additionally, as later described in reference to FIG. 8, the imaging device 40 may be implemented in an adjustable configuration that may allow the operator to adjust the rotational position or the proximal/distal position of the imaging device 40 relative to the distal jaw 14.

As previously discussed, the imaging head 84 may include one or more emitters 86 (e.g., light emitting diodes) disposed at a distal end portion 88 of the imaging device 40. In operation, the light emitted from the emitters 86 may illuminate the patient cavity 22, such that the image sensors 90 (e.g., complementary metal oxide semiconductor [CMOS] sensors) may capture details associated with the corresponding patient anatomy in the cavity 22. In this configuration, the imaging device 40 may provide for an improved visualization of the path of the mandible jaw 54b and the fixed jaw 54a over the jaw angle ϕ. By observing the image data captured by the imaging device(s) 40, an operator or surgeon may ensure that the target tissue 108a is effectively captured for connection with the suture 50 and the neighboring tissue 108b is avoided beyond the grasping extent of the distal jaw 14.

Referring now to FIGS. 8A, 8B, and 8C; the elongated surgical device 10 is shown demonstrating the imaging device 40 flexibly positioned relative to a distal jaw 14 via a positioning apparatus 120. In general, the positioning apparatus 120 may comprise a sleeve 122 or collar that allows the imaging device 40 to rotate or translate relative to the shaft forming the elongated probe 12. In this configuration, a user may engage a proximal interface 124 or grip portion and rotate the imaging device 40 about the longitudinal axis A_{L} over a rotation angle γ, such that the field of view FOV is repositioned about the distal jaw 14. In some implementations, the imaging device 40 may include an inertial sensor (e.g., accelerometer, inertial measurement unit, gyroscope, etc.), which may supply orientation signals (e.g., gravitational direction, field directions, etc.) to a controller 142 of the imaging system 140. In this configuration, the controller may track and offset the orientation of the field of view FOV of the imaging device 40 relative to gravity or another prevailing force in real time. In this configuration the controller 142 of the imaging system 140 (FIG. 9) may adjust an orientation of the image data 100, such that the anatomy or features in the cavity 22 are consistently presented relative to gravity or a predefined offset relative to gravity regardless of the rotation orientation γ of the imaging device 40 relative to the distal jaw 14.

At the distal end portion 12b of the elongated probe 12, the imaging device 40 may be connected to the sleeve 122 via a collar 126 or enclosure. The collar 126 may extend tangentially from the sleeve 122 positioning the imaging device 40 adjacent and parallel to the elongated probe 12. In this configuration, the imaging device may extend distally from the sleeve over an extension distance Dₑ. In this configuration, the imaging device 40 may be selectively positioned longitudinally along the elongated shaft over a telescoping portion 128. As illustrated in FIG. 8C, the extension distance Dₑ may provide for the imaging device 140 and the corresponding field of view FOV to be selectively positioned between a proximal end portion and a distal end portion of the distal jaw 14, allowing users to inspect about the perimeter of the distal jaw 14 to ensure accurate engagement of the elongated probe 12.

In operation, the positioning apparatus 120 may provide for the adjustment of the rotation angle γ and/or the longitudinal position P_{L} of imaging device 40 proximally and distally relative to the distal jaw 14. For example, in operation, the sleeve 122 may selectively extend over the telescoping portion 128 proximally or distally relative to the distal jaw 14. As shown in FIG. 8A, the imaging device 40 is positioned along the ventral surface of the elongated probe. In this position, the field of view FOV may clearly demonstrate the clearance of the fixed jaw 54a relative to the target tissue 108a and the neighboring tissue 108b. Additionally, the user or surgeon may extend the longitudinal position P_{L} of the imaging device 40 to verify the clearance of the neighboring tissue 108b relative to the fixed jaw 54a as shown in FIG. 8A or relative to the mandible jaw 54b as demonstrated in FIG. 8C. Further, as the imaging device 40 is rotated over the rotation angle γ about the elongated probe 12, the orientation of the image data 100 may remain in a constant orientation relative to gravity or an offset therefrom. Stated differently, the representations of the tissues or objects in the field of view FOV may maintain the same orientation (e.g., up, down) as the imaging device 40 is oriented about the distal jaw 14 over the rotation angle γ. In this way, the position of the field of view FOV may be adjusted relative to the distal jaw 14 to more clearly demonstrate the target tissue 108a and the neighboring tissue 108b while the orientation of the tissue 108 remains consistently oriented despite the rotating orientation of the imaging device 40.

As previously discussed, the communication cable 114 of the imaging device 40 may extend along the elongated probe 12 in the trough 116, which may be included in the sleeve 122 and connect to the imaging device 40 at the distal collar 126 or enclosure. In such implementations, the communication cable 114 may include an extension adapter or an expansion loop within the body forming the proximal interface 124 or grip portion. In this way, the sleeve 122 may be free to change the rotation angle γ about the elongated probe 12 and translate proximally and distally along the longitudinal axis A_{L} varying the longitudinal position P_{L} to adjust the field of view of the imaging device 40 relative to the distal jaw 14. Such operation may ensure that the user is able to selectively view the surroundings about the distal jaw 14 or other distal features that may be implemented distally on the elongated probe 12.

Referring now to FIG. 9, a block diagram of the imaging system 140 is shown. In various implementations, the system 140 may correspond to a video control console in communication with the surgical scope 36 and the imaging device 40. The system 140 may further be in communication with various surgical tools via a controller 142. The surgical scope 36 may correspond to various devices including an endoscope, laparoscope, arthroscope, etc. The imaging device 40 may correspond to a chip-on-wire imaging device comprising the control circuitry 82 incorporated in the imaging head 84 at the distal end portion of the elongated probe 12. As demonstrated, the surgical scope 36 and the imaging device 40 may be in communication with the controller 142 via a communication interface. Though shown connected via a conductive connection, the communication interface may correspond to a wireless communication interface operating via one or more wireless communication protocols (e.g., Wi-Fi, 802.11 b/g/n, etc.).

In various implementations, the imaging head 84 may incorporate one or more of the emitters 86, which may correspond to various light emitters configured to generate light in the visible range, the near infrared range, or various wavelengths. In various implementations, the emitters 86 may include light emitting diodes (LEDs), laser diodes, or other lighting technologies. The image sensor(s) 90 may correspond to various sensors and configurations comprising, for example, complementary metal-oxide semiconductor (CMOS) sensors, or similar sensor technologies. In some implementations, the imaging device 40 may include an inertial sensor (e.g., accelerometer, inertial measurement unit, gyroscope, magnetometer etc.), which may supply orientation signals (e.g., gravitational direction, field directions, etc.) to the controller 142. In this configuration, the controller 142 may track and offset the orientation of the field of view FOV of the imaging device 40 relative to gravity or another prevailing force in real time or with minimal delay associated with a framerate (e.g., 30 frames per second [FPS], 60 FPS, 90 FPS) of the image feed captured by the image sensor(s) 90. In this configuration a controller 142 may adjust an orientation of the image data 100, such that the anatomy or features in the cavity 22 are consistently presented relative to gravity or a predefined offset relative gravity regardless of the rotation angle γ of the imaging device 40 about the elongated probe 12 and the distal jaw 14. Accordingly, the controller 142 of the imaging system may process image data responsive to the orientation of the image sensor 90 to present the features of the operating environment consistently with respect to gravity or other prevailing/detectable forces or signals. For example, while gravity is mentioned specifically, other prevailing signals (e.g., artificial or natural magnetic fields), radio frequency signals, or similar signals may be detected to consistently orient the image data relative to gravity or another direction of interest.

In various implementations, the imaging device 40 may comprise the control circuitry 82 configured to control the operation of image sensor(s) 90 and the emitter(s) 86 as well as process and/or communicate the image data to the controller 142. Additionally, the control circuitry 82 may be in communication with a user interface 144, which may include one or more input devices, indicators, displays, etc. The user interface 144 may provide for the control of the imaging device 40 including the activation of one or more control routines. The user interface 144 may provide for the selection, adjustment, or toggling of one or more of the image feeds associated with the operation of the imaging device 40 and/or the scope 36. The control circuitry 82 may be implemented by various forms of controllers, microcontrollers, application-specific integrated controllers (ASICs), and/or various control circuits or combinations.

The controller 142 or system controller may comprise a processor 146 and a memory 148. The processor 146 may include one or more digital processing devices including, for example, a central processing unit (CPU) with one or more processing cores, a graphics processing unit (GPU), digital signal processors (DSPs), field programmable gate arrays (FPGAs), application-specific integrated circuits (ASICs) and the like. In some configurations multiple processing devices are combined into a System on a Chip (SoC) configuration while in other configurations the processing devices may correspond to discrete components. In operation, the processor 146 executes program instructions stored in the memory 148 to perform various operations related to the operation of the imaging system 140 as well as one or more surgical control consoles in communication with the controller 142.

The memory 148 may comprise one or more data storage devices including, for example, magnetic or solid-state drives and random access memory (RAM) devices that store data. The memory 148 may include one or more stored program instructions, object detection templates, image processing algorithms, etc. In various implementations, the controller 142 may correspond to a display or video controller configured to output formatted image data to one or more display devices 150. In such applications, the controller 142 may include one or more formatting circuits 154, which may process the image data received from the imaging device 40 and/or the surgical scope 36, communicate with the processor 146, and process the image data for presentation on the one or more display devices 150. The formatting circuits 154 may include one or more signal processing circuits, analog-to-digital converters, digital-to-analog converters, etc. The user interface 144 of the controller 142 may be in the form of an integrated interface (e.g., a touchscreen, input buttons, an electronic display, etc.) or may be implemented by one or more connected input devices (e.g., a tablet) or peripheral devices (e.g., keyboard, mouse, foot pedal, etc.).

As shown, the controller 142 may also be in communication with an external device or server 140, which may correspond to a network, local or cloud-based server, device hub, central controller, or various devices that may be in communication with the controller 142 and, more generally, the imaging system 140 via one or more wired (e.g., serial, Universal Serial Bus (USB), Universal Asynchronous Receiver/Transmitter (UART), etc.) and/or wireless communication interfaces (e.g., a ZigBee, an Ultra-Wide Band (UWB), Radio Frequency Identification (RFID), infrared, Bluetooth®, Bluetooth® Low Energy (BLE), Near Field Communication (NFC), etc.) or similar communication standards or methods. For example, the controller 142 may receive updates to the various modules and routines as well as communicate sample image data from the imaging device 40 to a remote server for improved operation, diagnostics, and updates to the imaging system 140. The user interface 144, the external server 140, and/or a surgical control console may be in communication with the controller 142 via one or more I/O circuits 144. The I/O circuits 144 may support various communication protocols including, but not limited to, Ethernet/IP, TCP/IP, Universal Serial Bus, Profibus, Profinet, Modbus, serial communications, etc.

According to some aspects of the disclosure, a surgical apparatus includes an elongated probe with a distal jaw and a proximal handle portion. An imaging device is in connection with a portion of the elongated probe proximate to the distal jaw, wherein the imaging device comprises a camera configured to capture image data depicting a grasping path of the distal jaw.

According to various aspects, the disclosure may implement one or more of the following features or configurations in various combinations:
the distal jaw forms inner grasping surfaces and outer jaw surfaces opposite the inner grasping surfaces;
the imaging device is in connection with one of the outer jaw surfaces;
the distal jaw is formed by a fixed jaw in connection with a mandible jaw, and the imaging device is in connection with the elongated probe on the outer jaw surface proximate to the fixed jaw;
the outer jaw surfaces form a dorsal surface from which the mandible jaw extends and a ventral surface forming the fixed jaw, wherein the imaging device is in connection with the ventral surface;
a retractable needle selectively extended from one of the inner grasping surface of the distal jaw;
the retractable needle is configured to pass a suture across opposing sides of the distal jaw;
the retractable needle is selectively deployed via an actuator assembly in connection with the proximal handle portion;
a marker positioned on one of the outer jaw surfaces in alignment with the retractable needle;
the marker is positioned on the outer jaw surface within the field of view demonstrating the marker and indicating a needle position of the retractable needle on the inner grasping surface opposite the outer jaw surface; and/or
the imaging device captures image data in a field of view distally directed from the elongated probe.

According to another aspect of the disclosure, a method for passing a suture through tissue relative to a feature or landmark includes the steps of inserting a distal jaw of an elongated probe into a patient cavity; capturing image data in a first field of view with an imaging device in connection with the elongated probe proximate to the distal jaw; viewing the feature or landmark from an outer jaw surface of the distal jaw in the first field of view; positioning the elongated probe in the patient cavity aligning a marker positioned along the outer jaw surface of the distal jaw with the feature or landmark demonstrated in the first field of view; and deploying a suture passing needle through patient tissue captured between opposing inner jaw surfaces of the distal jaw.

According to various aspects, the disclosure may implement one or more of the following features or configurations in various combinations:
the suture passing needle extends from one of the inner jaw surfaces aligned with the marker opposite the outer jaw surface;
the feature or landmark comprises surgical anchor, anatomic feature or defect, or a portion of a surgical construct;
the patient tissue comprises a rotator cuff tendon;
the suture passing needle extends from the inner jaw surface of a fixed jaw and passes the suture to a mandible jaw of the distal jaw;
the patient tissue in the patient cavity comprises target tissue and neighboring tissue;
the method distinguishes between the target tissue and neighboring tissue on a ventral side of the outer jaw surface of the distal jaw based on the image data; and/or
viewing the patient cavity from a second field of view with a surgical scope, wherein the feature or landmark is occluded from view in the second field of view by the patient tissue.

According to yet another aspect of the disclosure, a surgical apparatus comprises an elongated probe comprising a distal jaw and a proximal handle portion. The distal jaw forms inner grasping surfaces and outer jaw surfaces opposite the inner grasping surfaces. The outer jaw surfaces form a dorsal surface from which the mandible jaw extends and a ventral surface forms the fixed jaw. An imaging device is in connection with a portion of the elongated probe proximate to the distal jaw, wherein the imaging device comprises a camera configured to capture image data depicting a grasping path of the distal jaw, wherein the imaging device is in connection with the ventral surface.

According to various aspects, the disclosure may implement one or more of the following features or configurations in various combinations:
a retractable needle selectively extended from one of the inner grasping surface of the distal jaw; and/or
a marker positioned on one of the outer jaw surfaces in alignment with the retractable needle.

According to another aspect of the disclosure, a surgical imaging apparatus comprises an elongated probe including a proximal end portion and a distal end portion. An imaging device is in connection with a portion of the elongated probe at the distal jaw portion. The imaging device comprises a camera configured to capture image data in a field of view and a positioning mechanism comprising an interface surface operably connected to the proximal end portion and in connection with the imaging device at the distal end portion, wherein the positioning mechanism adjusts a rotational position about the elongated probe or a longitudinal position of the imaging device along the elongated probe.

According to various aspects, the disclosure may implement one or more of the following features or configurations in various combinations:
an inertial sensor operably connected with the imaging device and configured to detect orientation data indicating changes in the rotational position of the camera about the elongated probe;
a controller configured to receive the image data and the orientation data and adjust an image orientation of the image data in response to the orientation data;
the controller is further configured to adjust the image orientation maintaining a horizon of a scene depicted in the image data in response to changes in the rotational position of the imaging device about the elongated probe;
the positioning mechanism comprises a sleeve extending about at least a portion of the elongated probe operably connecting the imaging device with the interface surface;
the sleeve extends and retracts along a length of the elongated probe adjusting the longitudinal position of the imaging device in repose to a change in a position of the interface surface;
the scene depicted in the image data demonstrates a patient anatomy within a cavity accessed by the distal end portion of the elongated probe;
the elongated probe comprises a distal jaw and a proximal handle portion, wherein the image data depicts a grasping path of the distal jaw;
the positioning mechanism adjusts the rotational position about the elongated probe or the longitudinal position of the imaging device selectively aligning the field of view relative to the grasping path of the distal jaw; and/or
the positioning mechanism selectively adjusts the longitudinal position of the imaging device between a proximal end portion and a distal end portion of the distal jaw.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A surgical apparatus comprising:
an elongated probe comprising a distal jaw and a proximal handle portion; and
an imaging device in connection with a portion of the elongated probe proximate to the distal jaw, wherein the imaging device comprises a camera configured to capture image data depicting a grasping path of the distal jaw.

2. The surgical apparatus according to claim 1, wherein the distal jaw forms inner grasping surfaces and outer jaw surfaces opposite the inner grasping surfaces.

3. The surgical apparatus according to claim 2, wherein the imaging device is in connection with one of the outer jaw surfaces.

4. The surgical apparatus according to claim 3, wherein the distal jaw is formed by a fixed jaw in connection with a mandible jaw, and the imaging device is in connection with the elongated probe on the outer jaw surface proximate to the fixed jaw.

5. The surgical apparatus according to claim 2, wherein the outer jaw surfaces form a dorsal surface from which the mandible jaw extends and a ventral surface forming the fixed jaw, wherein the imaging device is in connection with the ventral surface.

6. The surgical apparatus according to claim 2, further comprising:
a retractable needle selectively extended from one of the inner grasping surface of the distal jaw.

7. The surgical apparatus according to claim 6, wherein the retractable needle is configured to pass a suture across opposing sides of the distal jaw.

8. The surgical apparatus according to claim 6, wherein the retractable needle is selectively deployed via an actuator assembly in connection with the proximal handle portion.

9. The surgical apparatus according to claim 6, further comprising:
a marker positioned on one of the outer jaw surfaces in alignment with the retractable needle.

10. The surgical apparatus according to claim 9, wherein the marker is positioned on the outer jaw surface within the field of view demonstrating the marker and indicating a needle position of the retractable needle on the inner grasping surface opposite the outer jaw surface.

11. The surgical apparatus according to claim 1, wherein the imaging device captures image data in a field of view distally directed from the elongated probe.

12. A method for passing a suture through tissue relative to a feature or landmark, the method comprising:
inserting a distal jaw of an elongated probe into a patient cavity;
capturing image data in a first field of view with an imaging device in connection with the elongated probe proximate to the distal jaw;
viewing the feature or landmark from an outer jaw surface of the distal jaw in the first field of view;
positioning the elongated probe in the patient cavity aligning a marker positioned along the outer jaw surface of the distal jaw with the feature or landmark demonstrated in the first field of view; and
deploying a suture passing needle through patient tissue captured between opposing inner jaw surfaces of the distal jaw.

13. The method according to claim 12, wherein the suture passing needle extends from one of the inner jaw surfaces aligned with the marker opposite the outer jaw surface.

14. The method according to claim 12, wherein the feature or landmark comprises surgical anchor, anatomic feature or defect, or a portion of a surgical construct.

15. The method according to claim 12, wherein the patient tissue comprises a rotator cuff tendon.

16. The method according to claim 12, wherein the suture passing needle extends from the inner jaw surface of a fixed jaw and passes the suture to a mandible jaw of the distal jaw.

17. The method according to claim 12, wherein the patient tissue in the patient cavity comprises target tissue and neighboring tissue, and the method further comprises:
distinguishing between the target tissue and neighboring tissue on a ventral side of the outer jaw surface of the distal jaw based on the image data.

18. The method according to claim 12, further comprising:
viewing the patient cavity from a second field of view with a surgical scope, wherein the feature or landmark is occluded from view in the second field of view by the patient tissue.

19. A surgical apparatus comprising:
an elongated probe comprising a distal jaw and a proximal handle portion, wherein the distal jaw forms inner grasping surfaces and outer jaw surfaces opposite the inner grasping surfaces and the outer jaw surfaces form a dorsal surface from which the mandible jaw extends and a ventral surface forming the fixed jaw; and
an imaging device in connection with a portion of the elongated probe proximate to the distal jaw, wherein the imaging device comprises a camera configured to capture image data depicting a grasping path of the distal jaw, wherein the imaging device is in connection with the ventral surface.

20. The surgical apparatus according to claim 19, further comprising:
a retractable needle selectively extended from one of the inner grasping surface of the distal jaw; and
a marker positioned on one of the outer jaw surfaces in alignment with the retractable needle.
